(19) United States
(12) Patent Application Publication
(10) Pub. No.: US 2006/0276886 A1
(43) Pub. Date: Dec. 7, 2006

(54) Ten-thousandths scale metal reinforced stent delivery guide sheath or restraint

(75) Inventors: William R. George, Santa Cruz, CA (US); Joseph Thomas Kavanagh, Mountain View, CA (US)

Correspondence Address: BOZICEVIC, FIELD & FRANCIS LLP (CARDIOMIND), 1900 UNIVERSITY AVENUE, SUITE 200, EAST PALO ALTO, CA, 94303, US

(73) Assignee: CardioMind, Inc.

(21) Appl. No.: 11/147,999

(22) Filed: Jun. 7, 2005

(51) Int. Cl. A61F 2/06

(52) U.S. Cl. 623/1.44

(57) ABSTRACT

A high-strength thin-walled tubular material having a lubricious polymer inner layer is disclosed. The tubing may be used in constructing delivery systems for radially expanding prostheses for use in the treatment of atherosclerosis in stenting procedures or a variety of other procedures.

### BACKGROUND OF THE INVENTION

Implants such as stents and occlusive coils have been used in patients for a wide variety of reasons. One of the most common “stenting” procedures is carried out in connection with the treatment of atherosclerosis, a disease which results in a narrowing and stenosis of body lumens, such as the coronary arteries. At the site of the narrowing (i.e., the site of a lesion) a balloon is typically dilatated in an angioplasty procedure to open the vessel. A stent is set in apposition to the interior surface of the lumen in order to help maintain an open passageway. This result may be effected by means of scaffolding support alone or by virtue of the presence of one or more drugs carried by the stent aiding in the prevention of restenosis.

Various stent designs have been developed and used clinically, but selfexpandable and balloon-expandable stent systems and their related deployment techniques are now predominant. Examples of self-expandable stents currently in use are the Magic WALLSTENT® stents and Radius stents (Boston Scientific). A commonly used balloon-expandable stent is the Cypher® stent (Cordis Corporation). Additional self-expanding stent background is presented in: “An Overview of Superelastic Stent Design,” Min. Invas Ther & Allied Technol 2002: 9(3/4) 235-246, “A Survey of Stent Designs,” Min. Invas Ther & Allied Technol 2002: 11(4) 137-147, and “Coronary Artery Stents: Design and Biologic Considerations,” Cardiology Special Edition, 2003: 9(2) 9-14, “Clinical and Angiographic Efficacy of a Self-Expanding Stent” Am Heart J 2003: 145(5) 868-874.

Because self-expanding prosthetic devices need not be set over a balloon (as with balloon-expandable designs), self-expanding stent delivery systems can be designed to a relatively smaller outer diameter than their balloon-expandable counterparts. As such, self-expanding stents may be better suited to reach the smallest vasculature or achieve access in more difficult cases.

To realize such benefits, however, there continues to be a need in developing improved stents and stent delivery systems. Problems encountered with known delivery systems include drawbacks ranging from failure to provide means to enable precise placement of the subject prosthetic, to a lack of space efficiency in delivery system design. Space inefficiency in system design prohibits scaling the systems to sizes as small as necessary to enable difficult access or small-vessel procedures (i.e., in tortuous vasculature or vessels having a diameter less than 3 mm, even less than 2 mm).

In contrast to other known designs, certain stent delivery systems developed by the assignee hereof (e.g., as described in U.S. patent application Ser. No. 10/792,684) are amenable to scaling to extremely small sizes. A number of these systems employ a tubular restraint for holding a stent in a collapsed configuration.

For tubular restraint or sheath based systems that can practicably be scaled to sizes having an outer diameter at the stent of less than about 0.018 inches, in-sheath or in-restraint stent forces can be quite high when superelastic (SE) Nitinol (NiTi alloy) is used for the stent. The reason for this stems from the fact that for a SE Nitinol self expanding stent sized to expand from so small a diameter to one able to treat a vessel between about 2.0 mm and 3.5 mm that the stent must be compressed to between about 10 and 20% of its full diameter. Where a shape memory alloy (SMA) Nitinol stent would remain in a collapsed state until heated, with a SE Nitinol stent, expansion is limited only by the restraint itself.

Another consideration in creating small diameter delivery systems (i.e., sub 0.018 inch diameter) is the result that the constituent components are very thin, delicate or fragile. The delicacy of the features, in conjunction with the high in-sheath or in-restraint stent forces accompanying SE Nitinol stent use presents numerous problems concerning sheath or restraint design.

For example, the tubular member must have sufficient strength to avoid problematic deformation by the stent (either upon initial action or over time due to material creep), that can otherwise result in an interlocking relationship between the members. However, selection of stronger materials may exclude the use of low-friction materials.

U.S. Pat. No. 6,689,120 (Gerdts) addresses this issue for a delivery catheter sheath of a much larger scale than used in the present invention. Specifically, the sheath in the patent incorporates a reinforcing metal braid into the wall of the tubular body. To provide a “reduced profile” delivery system, a flat ribbon braid is used in lieu of round wire. The structure includes an inner layer (optionally, polytetrafluoroethylene—PTFE) fused to an outer layer (optionally a polyether ester or polymeric amide) with the reinforcing coil there between. The construction is provided to allow a relatively compact delivery catheter with torquability and pushablity characteristics comparable to more bulky devices. Yet, the thinnest exemplary wall given for this three-layer structure is about 0.004 inches.

As such, the construction techniques described in the '120 patent are not believed to be suitable for producing a reinforced sheath or restraint between about 0.00075 and about 0.002 inches thick. Since stent delivery systems having an overall diameter of less than about 0.018 inches may require such thin-walled tubing in order to provide adequate space for a stent and components internal thereto, there exists a need for approach to tubular member construction that can be employed to reach these sizes.

For small diameter tubing (e.g.., tubing having an inner diameter of about 0.015 inches), it is well recognized that internal coating by way of flowing lubricious polymer therethrough cannot presently be accomplished. Material that is viscous enough to provide a thick/durable coating in larger diameter tubing simply undergoes plug flow when coating is attempted. Less viscous material only produces a thin/friable or delicate layer offering little utility under high stress contact dynamic friction situations.

Accordingly, there exists a need for a means of manufacturing small diameter/thin walled metal tubing with a lubricious polymer layer provided therein. The present invention offers a number of approaches in this regard.

### SUMMARY OF THE INVENTION

The present invention addresses the need for a reduced wall thickness and/or diameter reinforced sheath/restraint by way of providing a metal layer or metalized portion set over a lubricious polymeric layer (e.g., PTFE). A two-layer construction with metal as an outer layer may be preferred. Such a structure can be made by mechanically interlocking or gluing the parts together. In addition, such a structure may be provided by coating a polymeric tube with a metallic layer.

However constructed, it is also contemplated that the metal may be coated with another polymeric layer for the purpose of biocompatibility or another reason such as stabilizing the metal coating from fracture or flaking. Still, any such coating will generally be quite thin in order that the thickness of the composite structure does not exceed about 0.002 inches.

At larger sizes, thicker wall sections can be more cost effectively produced in other manners. For wall sections of less than about 0.002 inches, the construction approaches of the present invention offer particular benefit. However, in some applications they may be advantageously applied to thicker wall/larger structures.

A coating approach to construction is desirable from the perspective of providing an imperforate outer shell or tubular body. However, the alloys that can be employed may be limited.

Another approach for producing the subject hybrid structures employs mechanically interlocking an inner polymeric tube with an outer metal tube. This may be accomplished in a number of manners. In each, the metal tube will include a plurality of openings serving to mechanically interlock with tubes together. A thin-walled polymer tube is set within the metal tube. By expanding the polymer tube (e.g., under air pneumatic or hydraulic pressure) or collapsing the metal tube upon the polymer (e.g., by crimping or shape recovery of an expanded tube SMA tube) an interference fit between the members can be produced.

Yet another approach involves gluing or tacking the tubular polymeric liner inside the outer tubular member. Again, the tubular metal structure will include a plurality of holes. Except instead of offering a mechanical interlock, the windows offer sites for gluing or tacking the tubular members together. The glue employed may be a cyanoacrylate. The means of tacking may involve bonding/welding fill material within the holes to the inner polymer layer. Another approach to tacking may involve heating the polymer by hot air, electrically or otherwise to melt through the exposed sections of polymer so that they bead-up around the edge of their respective openings. To insure a smooth inner bore when employing such an approach, the inner lumen may by occupied by a mandrel during bead formation or post-process reamed or machined-out. Yet another approach involves heat shrinking a polymer tube over the metal tube such that it fills its holes, bonding with the interior polymer tube. The outer tube may be left in place or skived off, leaving only thin inner tube and sections of the outer tube bonded thereto set within the metal tube windows to form an interlock. Yet another possibility employs a heated metal tube crimped upon the polymer layer set upon a mandrel, in which the pressure and/or heat is sufficient to cause the polymer to flow and fill the openings in the metal tubing.

The metal tube used in this interlocking construction approach will typically have a wall thickness from about 0.00025 to about 0.001 inches. The tubing may comprise stainless steel, Ti, NiTi, another Ti alloy, NiCo, another Ni alloy, CoCr, PtIr, PtW, BeCu or others of relatively high strength or other desirable properties such as high radiopacity, etc.

Its openings may be formed in the tubing by laser machining, electrical discharge machining (EDM) or otherwise. The pattern selected for the cutting may be as described in U.S. Pat. No. 6,428,489 (Jacobsen) more simply staggered or close-packed circular holes, etc. The pattern selected may offer assistance with flex and/or torque transmission characteristics. Various qualities of the pattern, such as maximized hole size may improve the interlock or interconnection between the inner and outer tubes.

In producing this variation of the invention, the polymeric tube is fed into or through the metal tube. To do so, the tube to be employed in the composite structure may be configured with sufficient column strength to accomplish this alone. Alternatively, the tube may be fed in over a mandrel that is removed once the other members are affixed to one another.

In another approach, a sacrificial tube or mandrel is provided upon which the desired polymer for the final composite construction is set. The outer polymer layer (often PTFE) may be sprayed-on, the product of dip-coating or another deposition approach. In which case, it is feasible to offer wall thickness to the inner polymer tubular layer of as little as about 0.0001 to about 0.0002 inches. After connection of this layer to the metal tube, the inner material (be it polyimide, steel, aluminum, or another material) is etched out.

In this manner, an extremely thin, yet durable lubricious polymer layer can be provided within the metal tube. Such an approach is useful in light of the difficulties inherent to drawing-down polymeric tubing to wall thicknesses of about 0.0005 inches or less. In any case, polymer tube formation by material deposition and removal may be preferred for reason of the dimensional consistency offered by a sprayed, dipped or otherwise built-up coating.

Returning to the first approach disclosed above for constructing the subject tubing, a PVD (Physical or Plasma Vapor Deposition) process may be employed in covering a polymeric tubular member to generate the composite structure. PVD may be used exclusively, where a material such as stainless steel, nickel, titanium or a titanium alloy (i.e., a material having sufficient strength—as elaborated upon below) is deposited upon the polymer. In the alternative, a non-structural but highly conductive substance or metal (e.g., aluminum, copper, gold, silver, platinum, palladium alloys thereof, etc.) can first be deposited on the polymeric member. In which case, electroplating or electroforming will then be employed to deposit the desired structural layer upon the conductive layer.

Of course, should the polymer substrate upon which metal is to be deposited possess sufficient conductivity (as possibly provided by the inclusion of conductive material therein, e.g., carbon particulates, nanotubes, etc.) then a PVD step might be avoided altogether. In any case, suitable metal shell materials include the aforementioned stainless steel, Ti, NiTi, another Ti alloy, Ni, Nickel Sulfamate, High Phosphate Nickel, NiCo, another Ni alloy, CoCr or others.

If a non-structural layer is employed for electrical conduction to facilitate electroplating or electroforming, the material thickness may be as little as several angstroms. Thicker layers may be employed, but employing expensive PVD processes to deposit a non-structural layer is not efficient. Yet, by using stainless steel, or alloyed gold, the thin “conductive” layer may also serve a structural purpose.

As for the primary (or singular) structural shell formed directly over the polymer or a more conductive metallic layer, it is formed by one or more high-strength metals deposited in one or more layers. The material will generally range from about 0.0002 to about 0.001 inches thick. More preferably, it will be between about 0.0004 and about 0.0008 inches thick.

In preparation for the deposition or plating, it may be desired or necessary to pre-treat the polymeric substrate. When PTFE is to be employed, a Sodium-based etchant that strips or neutralizes fluorine content from the outer portion of the structure may be employed. Such etchants are typically employed in preparing an adequate bonding surface for and/or between PTFE components. Other means of pretreating the surface, such as plasma etching, may also be employed.

It is contemplated that the plastic substrate upon which the metal is set be originally provided in the form of tubing. In which case, it may be necessary to draw-down larger diameter tubing to reach desirable wall thicknesses of between about 0.00025 and 0.0015 inches. To ensure dimensional stability, it may be desired that the tubing be set or remain on a mandrel for subsequent processing.

As described above, the polymeric layer may alternatively be coated or formed upon a sacrificial mandrel. After the metal of the composite tube is then deposited upon the polymer layer to retain, the inner material (e.g., polyimide, aluminum) will be etched out leaving the metal layer set only upon the remaining polymer layer.

However constructed, when tubing according to the present invention is employed in a stent delivery system, the polymeric layer serves to reduce frictional forces between the stent and overlying tube upon withdrawal of the latter from the stent. The metal coating offers both tensile and hoop strength to the composite structure.

As described above, the construction of the invention may vary in terms of wall thickness, diameter, material composition and/or manufacture approach. Still further, the amount of coverage of the polymeric tube or pattern of coverage may be varied. Various exemplary patterns are provided below. These examples are provided in order to provide increased flexibility or conformability of the tube to stresses, while still offering desirable axial and/or radial strength. Notwithstanding, other considerations may be taken into account for a given design.

One aspect of the present invention includes delivery systems in which a sheath or distal restraint is made from the tubing described herein. Another aspect of the invention concerns the composite tubing itself. The tubing may find applications in medical device construction as hypotubing (e.g., as for a catheter body, a microlumen for a balloon) or in another field. Still further, the present invention includes the methods involved in use or the production of any such product.

### BRIEF DESCRIPTION OF THE DRAWINGS

Each of the figures diagrammatically illustrates aspects of the invention. Of these:

FIG. 1 shows a heart in which its vessels may be the subject of one or more angioplasty and stenting procedures;

FIG. 2A shows an expanded stent cut pattern as may be used in producing a stent according to a first aspect of the invention; FIG. 2B shows a stent cut pattern for a second stent produced according to another aspect of the present invention;

FIG. 3A shows an expanded stent cut pattern as may be used in producing a stent according to a first aspect of the invention; FIG. 3B shows a stent cut pattern for a second stent produced according to another aspect of the present invention;

FIGS. 4A-4L illustrate stent deployment methodology to be carried out with the subject delivery guide member;

FIG. 5 provides an overview of a delivery system incorporating a tubular member according to the present invention;

FIGS. 6A and 6B show partial cutaway perspective views tubular members according to the present invention;

FIG. 7 shows a perspective view of tubular member as provided according to either of FIGS. 6A or 6B at an intermediate stage of production;

FIGS. 8A-8F illustrate optional metalizing patterns for producing composite tubular structures according to the present invention;

FIG. 9 shows an example of an interlocking approach to composite tubular construction according to the present invention;

FIGS. 10A-10E are sectional views showing a variety of modes for interconnecting the tubular metal outer structure with the inner polymeric tube;

FIGS. 11A and 11B show alternate alternative cut-out patterns for the metallic tubular member;

FIG. 12 shows another example of an interlocking composite tubular construction illustrating multiple different feature zones; and

FIGS. 13A and 13B show yet another construction approach involving connecting the turns of a coiled ribbion for producing metal-reinforced tubular members according to the present invention.

In the figures, like elements in some cases are indicated by a related numbering scheme. Furthermore, variation of the invention from the embodiments pictured is, of course, contemplated.

### DETAILED DESCRIPTION OF THE INVENTION

Various exemplary embodiments of the invention are described below. Reference is made to these examples in a non-limiting sense. They are provided to illustrate more broadly applicable aspects of the present invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. All such modifications are intended to be within the scope of the claims made herein.

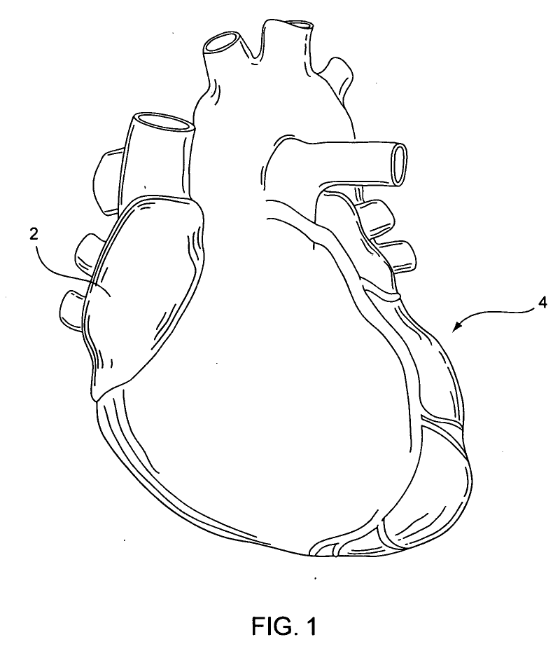

In light of this framework, FIG. 1 shows a heart 2 in which its vessels may be the subject of one or more angioplasty and/or stenting procedures. To date, however, significant difficulty or impossibility is confronted in reaching smaller coronary arteries 4. If a stent and a delivery system could be provided for accessing such small vessels and other difficult anatomy, an additional 20 to 25% coronary percutaneous procedures could be performed with such a system. Such potential offers opportunity for huge gains in human healthcare and a concomitant market opportunity in the realm of roughly $1 billion U.S. dollars—with the further benefit of avoiding loss of income and productivity of those treated.

Features of the present invention are uniquely suited for a system able to reach small vessels (though use of the subject systems s not limited to such a setting.) By “small” coronary vessels, it is meant vessels having a inside diameter between about 1.5 or 2 and about 3 mm in diameter. These vessels include, but are not limited to, the Posterior Descending Artery (PDA), Obtuse Marginal (OM) and small diagonals. Conditions such as diffuse stenosis and diabetes produce conditions that represent other access and delivery challenges which can be addressed with a delivery system according to the present invention. Other extended treatment areas addressable with the subject systems include vessel bifurcations, chronic total occlusions (CTOs), and prevention procedures (such as in stenting of vulnerable plaque).

Assuming a means of delivering one or more appropriately-sized stents, it may be preferred to use a drug eluting stent (DES) in such an application to aid in preventing restenosis. A review of suitable drug coatings and available vendors is presented in “DES Overview: Agents, release mechanism, and stent platform” a presentation by Campbell Rogers, Md. incorporated by reference in its entirety. However, bare-metal stents may be employed in the present invention.

While some might argue that the particular role and optimal usage of self expanding stents has yet to be defined, they offer an inherent advantage over balloon expandable stents. The latter type of devices produce “skid mark” trauma (at least when delivered uncovered upon a balloon) and are associated with a higher risk of end dissection or barotraumas caused at least in part by high balloon pressures and related forces when deforming a balloon-expandable stent for deployment.

Yet, with an appropriate deployment system, self-expanding stents may offer one or more of the following advantages over balloon-expandable models: 1) greater accessibility to distal, tortuous and small vessel anatomy—by virtue of decreasing crossing diameter and increasing compliance relative to a system requiring a deployment balloon, 2) sequentially controlled or “gentle” device deployment, 3) use with low pressure balloon pre-dilatation (if desirable) to reduce barotraumas, 4) strut thickness reduction in some cases reducing the amount of “foreign body” material in a vessel or other body conduit, 5) opportunity to treat neurovasculature—due to smaller crossing diameters and/or gentle delivery options, 6) the ability to easily scale-up a successful treatment system to treat larger vessels or vice versa, 7) a decrease in system complexity, offering potential advantages both in terms of reliability and system cost, 8) reducing intimal hyperplasia, and 9) conforming to tapering anatomy—without imparting complimentary geometry to the stent (though this option exists as well).

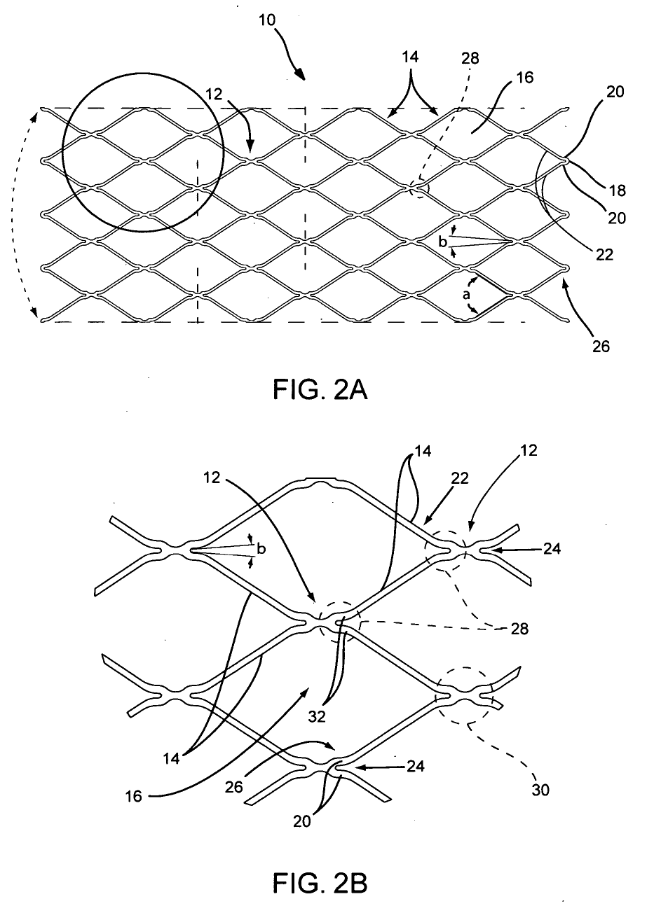

At least some of these noted advantages may be realized using a stent 10 as shown in FIG. 2A. The stent pattern pictured is well suited for use in small vessels. It may be collapsed to an outer diameter of about 0.018 inch (0.46 mm), or even smaller to about 0.014 inch (0.36 mm)—including the restraint/joint used to hold it down—and expanded to a size (fully unrestrained) between about 1.5 mm (0.059 inch) or 2 mm (0.079 inch) or 3 mm (0.12 inch) and about 3.5 mm (0.14 inch).

In use, the stent will be sized so that it is not fully expanded when fully deployed against the wall of a vessel in order to provide a measure of radial force thereto (i.e., the stent will be “oversized” as discussed above). The force will secure the stent and offer potential benefits in reducing intimal hyperplasia and vessel collapse or even pinning dissected tissue in apposition.

Stent 10 preferably comprises NiTi that is superelastic at or below room temperature and above (i.e., as in having an Af as low as 15 degrees C. or even 0 degrees C.). Also, the stent is preferably electropolished to improve biocompatibility and corrosion and fatigue resistance. The stent may be a DES unit. The drug can be directly applied to the stent surface(s), or introduced into pockets or an appropriate matrix set over at least an outer portion of the stent. The stent may be coated with gold and/or platinum to provide improved radiopacity for viewing under medical imaging.

For a stent able to collapse to an outer diameter of about 0.012 inches and expand to about 3.5 mm, the thickness of the NiTi is about 0.0025 inch (0.64 mm). Such a stent is designed for use in a 3 mm vessel or other body conduit, thereby providing the desired radial force in the manner noted above. Further information regarding radial force parameters in coronary stents may be noted in the article, “Radial Force of Coronary Stents: A Comparative Analysis,” Catheterization and Cardiovascular Interventions 46: 380-391 (1999), incorporated by reference herein in its entirety.

In one manner of production, the stent in FIG. 2A is laser or EDM cut from round NiTi tubing, with the flattened-out pattern shown wrapping around the tube as indicated by dashed lines. In such a procedure, the stent is preferably cut in its fully-expanded shape. By initially producing the stent to full size, the approach allows cutting finer details in comparison to simply cutting a smaller tube with slits and then heat-expanding/annealing it into its final (working) diameter. Avoiding post-cutting heat forming also reduces production cost as well as the above-reference effects.

Regarding the finer details of the subject stent, as readily observed in the detail view provided in FIG. 2B, necked down bridge sections 12 are provided between axially/horizontally adjacent struts or arms/legs 14, wherein the struts define a lattice of closed cells 16. Terminal ends 18 of the cells are preferably rounded-off so as to be atraumatic.

To increase stent conformability to tortuous anatomy, the bridge sections can be strategically separated or opened as indicated by the broken lines in FIG. 2A. To facilitate such tuning of the stent, the bridge sections are sufficiently long so that fully rounded ends 18 may be formed internally to the lattice just as shown on the outside of the stent if the connection(s) is/are severed to separate adjacent cells 16. Whether provided as ends 18 or adjoined by a bridge section 12, junction sections 28 connect circumferentially or vertically adjacent struts (as illustrated). Where no bridge sections are provided, the junction sections can be unified between horizontally adjacent stent struts as indicated in region 30.

The advantage of the optional double-concave profile of each strut bridge 12 is that it reduces material width (relative to what would otherwise be presented by a parallel side profile) to improve flexibility and thus trackability and conformability of the stent within the subject anatomy while still maintaining the option for separating/breaking the cells apart.

Further optional features of stent IO are employed in the cell end regions 18 of the design. Specifically, strut ends 20 increase in width relative to medial strut portions 22. Such a configuration distributes bending (during collapse of the stent) preferentially toward the mid region of the struts. For a given stent diameter and deflection, longer struts allow for lower stresses within the stent (and, hence, a possibility of higher compression ratios). Shorter struts allow for greater radial force (and concomitant resistance to a radially applied load) upon deployment.

In order to increase stent compliance so that it collapses as much as possible, accommodation is made for the stiffer strut ends 20 provided in the design shown in FIG. 2A. Namely, the gap 24 between the strut ends 22 is set at a smaller angle as if the stent were already partially collapsed in that area. Thus, the smaller amount of angular deflection that occurs at ends 20 can bring the sections parallel (or nearly so) when the strut medial portions 22 are so-arranged. In the variation of the invention in FIG. 2A, radiused or curved sections 26 provide a transition from a medial strut angle α (ranging from about 85 degrees to about 60 degrees) to an end strut angle β (ranging from about 30 to about 0 degrees) at the strut junctions 28 and/or extensions therefrom.

In addition, it is noted that gap 24 an angle β may actually be configured to completely close prior to fully collapsing angle α. The stent shown is not so-configured. Still, the value of doing so would be to limit the strains (and hence, stresses) at the strut ends 22 and cell end regions 18 by providing a physical stop to prevent further strain.

In the detail view of FIG. 2B, angle β is set at 0 degrees. The gap 24 defined thereby by virtue of the noticeably thicker end sections 20 at the junction result in very little flexure along those lever arms. The strut medial portions are especially intended to accommodate bending. In addition, a hinging effect at the corner or turn 32 of junction section 28 may allow the strut to swing around angle α to provide the primary mode for compression of the stent.

The stent pattern shown in FIG. 3A and detailed in FIG. 3B offers certain similarities as well as some major differences from the stent pattern presented in FIGS. 2A and 2B. As in the variation above, stent 40 includes necked down bridge sections 42 provided between adjacent struts or arms/legs 44, wherein the struts define a lattice of closed cells 46. In addition, terminal ends 48 of the cells are preferably rounded-off so as to be atraumatic.

Furthermore, the bridge sections 42 of stent 40 can be separated for compliance purposes. In addition, they may be otherwise modified (e.g., as described above) or even eliminated. Also, in each design, the overall dimensions of the cells and indeed the number of cells provided to define axial length and/or diameter may be varied (as indicated by the vertical and horizontal section lines in FIG. 3A).

Like the previous stent design, strut ends 50 may offer some increase in width relative to medial strut portions 52. However, as shown in FIG. 3B, as compared to FIG. 2B, the angle β is relatively larger. Such a configuration is not concerned with developing a hinge section and a relatively stiffer outer strut section. Instead, angle β in the FIG. 3A/3B design is meant to collapse and the strut ends are meant to bend in concert with the medial strut portions so as to essentially straighten-out upon collapsing the stent, generally forming tear-drop spaces between adjacent struts. This approach offers a stress-reducing radius of curvature where struts join, and maximum stent compression.

The “S” curves defined by the struts are produced in a stent cut to a final or near final size (as shown in FIGS. 3A and 3B). The curves are preferably determined by virtue of their origination in a physical or computer model that is expanded from a desired compressed shape to the final expanded shape. So derived, the stent can be compressed or collapsed under force to provide an outer surface profile that is as solid or smooth and/or cylindrical as possible or feasible.

Such action is enabled by distribution of the stresses associated with compression to generate stains to produce the intended compressed and expanded shapes. This effect is accomplished in a design unaffected by one or more expansion and heat setting cycles that otherwise deteriorate the quality of the superelastic NiTi stent material. Further details regarding the “S” stent design and alternative stent constructions as may be used in the present invention are disclosed in U.S. Provisional Patent Application Ser. No. 60/619,437, entitled, “Small Vessel Stent Designs”, filed Oct. 14, 2004 and incorporated herein by reference in its entirety. In the case of each of the above stent designs, by utilizing a stent design that minimizes problematic strain (and in the latter case actually uses the same to provide an improved compressed profile), very high compression ratios of the stent may be achieved from about 5× to about 10× or above.

Delivery systems according to the present invention are advantageously sized to correspond to existing guidewire sizes. For example, the system may have about an 0.014 (0.36 mm), 0.018 (0.46 mm), 0.022 (0.56 mm), 0.025 (0.64 mm) inch crossing profile. Of course, intermediate sizes may be employed as well, especially for full-custom systems. Still further, it is contemplated that the system sizing may be set to correspond to French (FR) sizing. In that case, system sizes contemplated range at least from about 1 to about 2 FR, whereas the smallest known balloon-expandable stent delivery systems are in the size range of about 3 to about 4 FR. In instances where the overall device crossing profile matches a known guidewire size, they may be used with off-the-shelf components such as balloon and microcatheters.

At least when produced in the smallest sizes (whether in an even/standard guidewire or FR size, or otherwise), the system enables a substantially new mode of stent deployment in which delivery is achieved through an angioplasty balloon catheter or small microcatheter lumen. Further discussion and details of “through the lumen” delivery is presented in U.S. Patent Application Ser. No. 10/746,455 “Balloon Catheter Lumen Based Stent Delivery Systems” filed on Dec. 24, 2003 and its PCT counterpart US2004/008909 filed on Mar. 23, 2004, each incorporated by reference in its entirety.

In larger sizes, (i.e., up to about 0.035 inch crossing profile or more), the system is most applicable to peripheral vessel applications as elaborated upon below. Yet, even in “small vessel” cases or applications (where the vessel to be treated has a diameter up to about 3.0 mm), it may also be advantageous to employ a stent delivery system sized at between about 0.022 to about 0.025 inch in diameter. Such a system can be used with catheters compatible with 0.022 and/or 0.025 inch diameter guidewires.

While such a system may not be suitable for reaching the very smallest vessels, this variation of the invention is quite advantageous in comparison to known systems in reaching the larger of the small vessels (i.e., those having a diameter of about 2.5 mm or larger). By way of comparison, among the smallest known over-the-guidewire delivery systems are the Micro-Driver™ by Medtronic and Pixel™ systems by Guidant. These are adapted to treat vessels between 2 and 2.75 mm, the latter system having a crossing profile of 0.036 inches (0.91 mm). A system described in U.S. Patent Publication No. 2002/0147491 for treating small vessels is supposedly capable of downsizing to 0.026 inch (0.66 mm) in diameter. Furthermore, because the core member of the subject device can be used as a guidewire (in one fashion or another) after stent delivery, the present invention offers further advantages in use as elaborated upon below.

As referenced above, it may be desired to design a variation of the subject system for use in deploying stents in larger, peripheral vessels, biliary ducts or other hollow body organs. Such applications involve a stent being emplaced in a region having a diameter from about 3.5 to 13 mm (0.5 inch). In which case, a 0.035 to 0.039 inch (3 FR) diameter crossing profile system is advantageously provided in which the stent expands (unconstrained) to a size between about roughly 0.5 mm and about 1.0 mm greater than the vessel or hollow body organ to be treated. Sufficient stent expansion is easily achieved with the exemplary stent patterns shown in FIGS. 2A/2B or 3A/3B.

Again, as a matter of comparison, the smallest delivery systems known to applicants for stent delivery in treating such larger-diameter vessels or biliary ducts is a 6 FR system (nominal 0.084 inch outer diameter), which is suited for use in an 8 FR guiding catheter. Thus, even in the larger sizes, the present invention affords opportunities not heretofore possible in achieving delivery systems in the size range of a commonly used guidewire, with the concomitant advantages discussed herein.

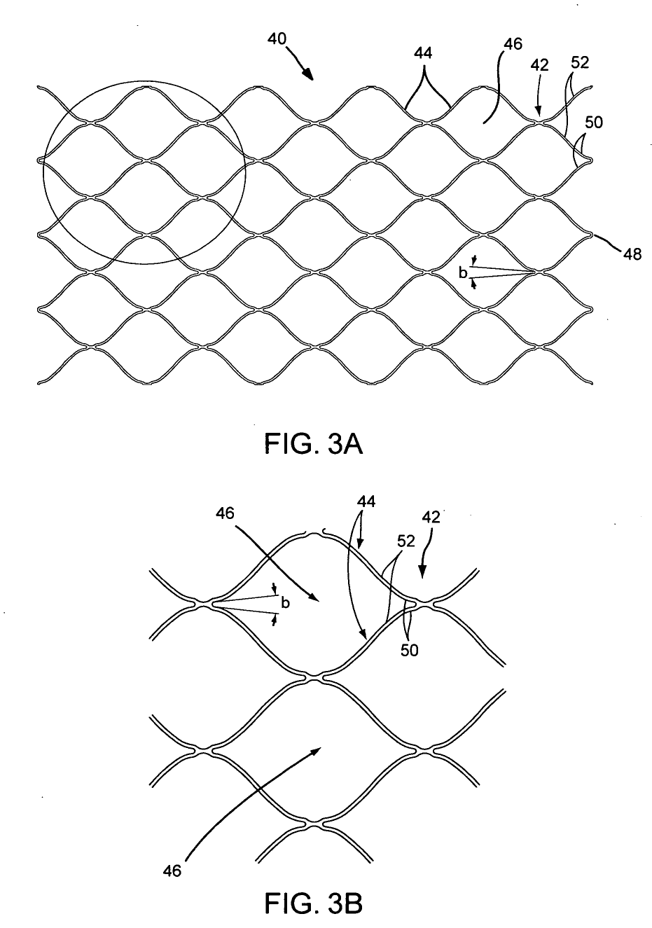

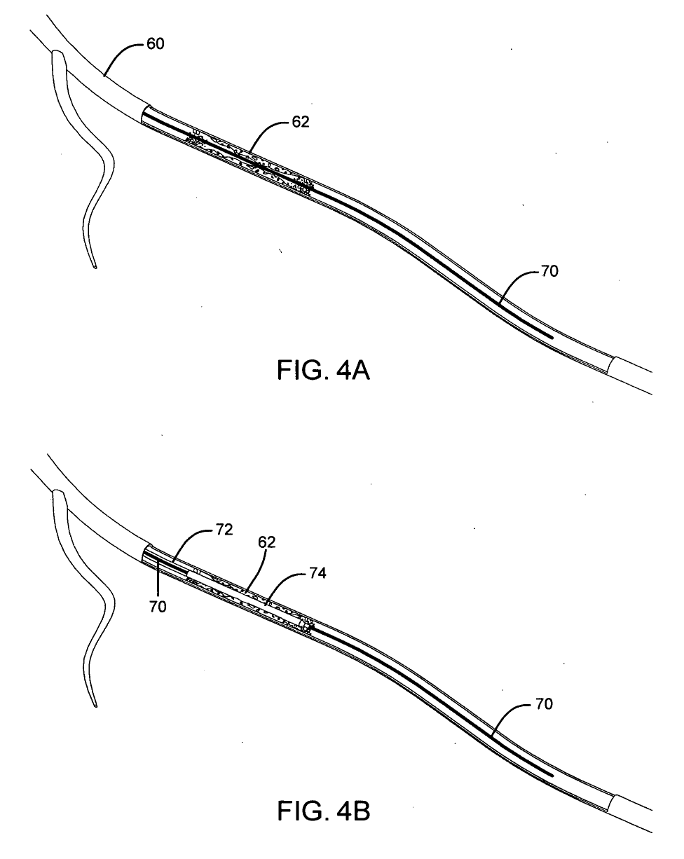

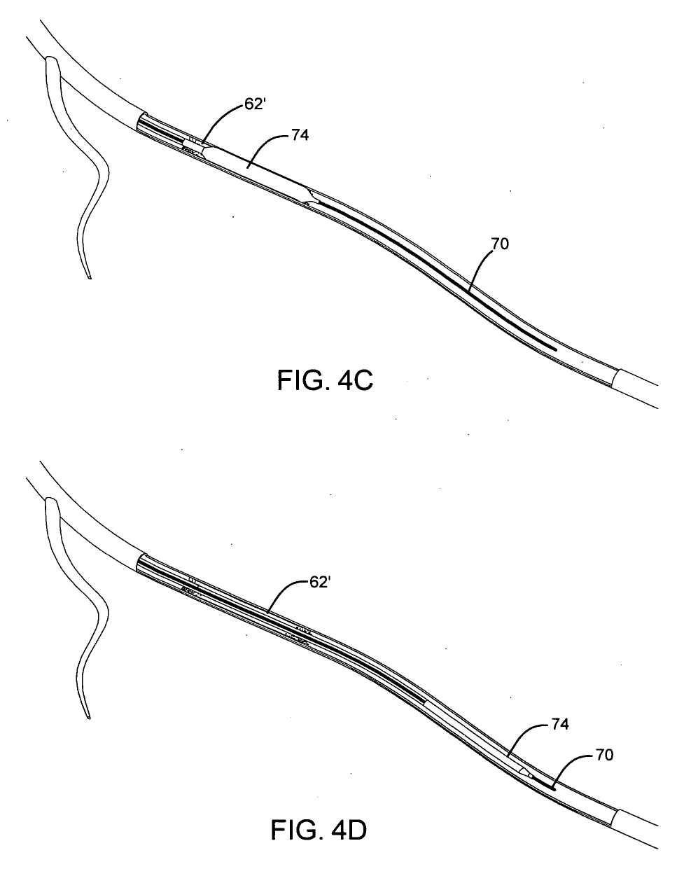

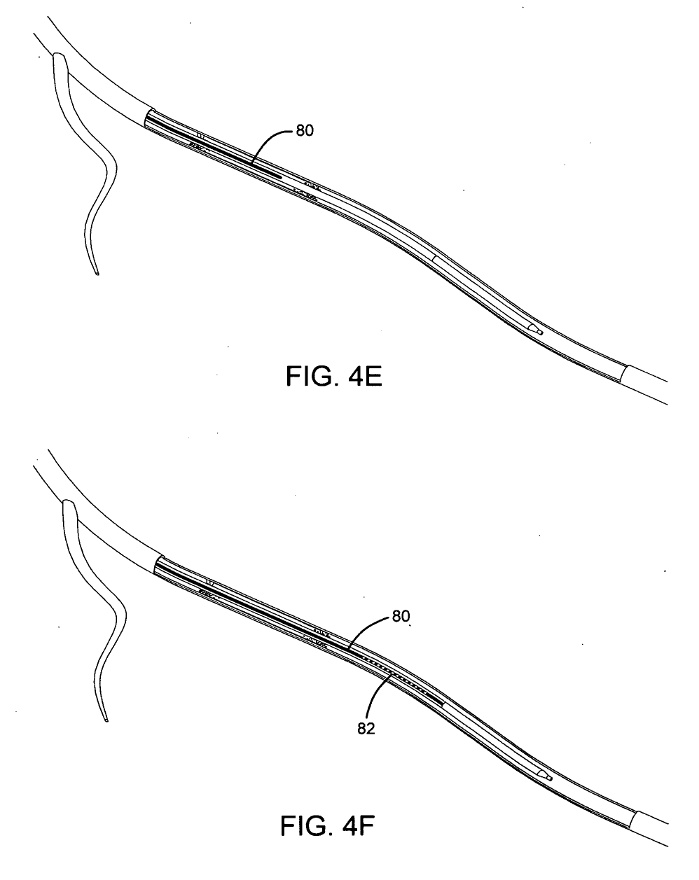

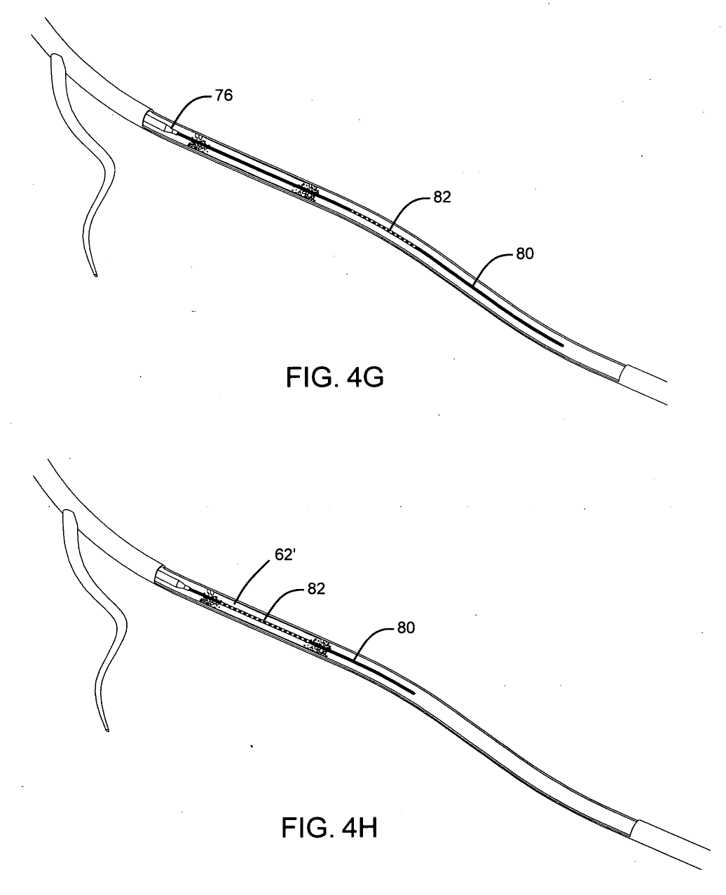

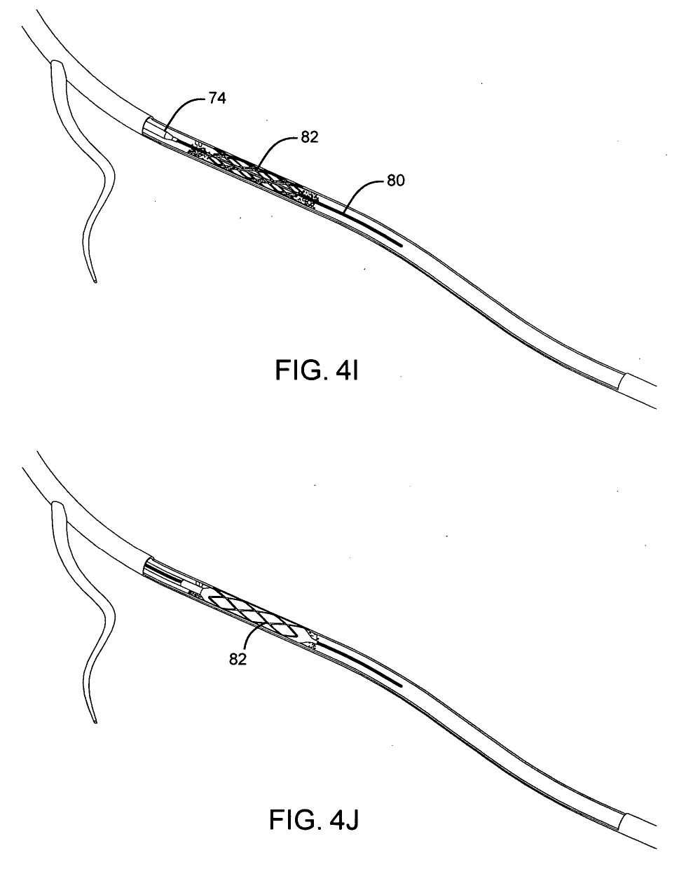

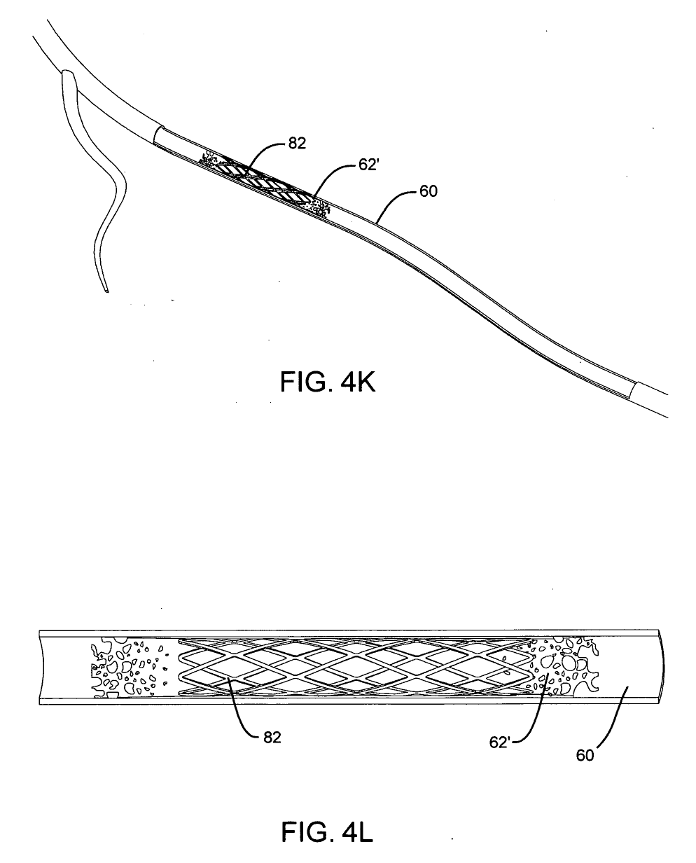

As for the manner of using the inventive system as optionally configured, FIGS. 4A-4L illustrate an exemplary angioplasty procedure. Still, the delivery systems and stents or implants described herein may be used otherwise—especially as specifically referenced herein.

Turning to FIG. 4A, it shows a coronary artery 60 that is partially or totally occluded by plaque at a treatment site/lesion 62. Into this vessel, a guidewire 70 is passed distal to the treatment site. In FIG. 4B, a balloon catheter 72 with a balloon tip 74 is passed over the guidewire, aligning the balloon portion with the lesion (the balloon catheter shaft proximal to the balloon is shown in cross section with guidewire 70 therein).

As illustrated in FIG. 4C, balloon 74 is expanded (dilatated or dialated) in performing an angioplasty procedure, opening the vessel in the region of lesion 62. The balloon expansion may be regarded as “predilatation” in the sense that it will be followed by stent placement (and optionally) a “postdilatation” balloon expansion procedure.

Next, for compatible systems (i.e., systems able to pass through a balloon catheter lumen) the balloon is at least partially deflated and passed forward, beyond the dilate segment 62′ as shown in FIG. 4D. At this point, guidewire 70 is removed as illustrated in FIG. 4E. It is exchanged for a delivery guide member 80 carrying stent 82 as further described below. This exchange is illustrated in FIGS. 4E and 4F.

However, it should be appreciated that such an exchange need not occur. Rather, the original guidewire device inside the balloon catheter (or any other catheter used) may be that of item 80, instead of the standard guidewire 70 shown in FIG. 4A. Thus, the steps depicted in FIGS. 4E and 4F (hence, the figures also) may be omitted.

Alternatively, the exchange of the guidewire for the delivery system may be made before the dilatation step. Yet another option is to exchange the balloon catheter used for predilatation for a fresh one to effect postdilatation.

In addition, there may be no use in performing the step in FIG. 4D of advancing the balloon catheter past the lesion, since such placement is merely for the purpose of avoiding disturbing the site of the lesion by moving a guidewire past the same. FIG. 4G illustrates the next act in either case. Particularly, the balloon catheter is withdrawn so that its distal end 76 clears the lesion. Preferably, delivery guide 80 is held stationary, in a stable position. After the balloon is pulled back, so is delivery device 80, positioning stent 82 where desired. Note, however, that simultaneous retraction may be undertaken, combining the acts depicted in FIGS. 4G and 4H. Whatever the case, it should also bet appreciated that the coordinated movement will typically be achieved by virtue of skilled manipulation by a doctor viewing one or more radiopaque features associated with the stent or delivery system under medical imaging.

Once placement of the stent across from dilated segment 62′ is accomplished, stent deployment commences. The manner of deployment is elaborated upon below. Upon deployment, stent 82 assumes an at least partially expanded shape in apposition to the compressed plaque as shown in FIG. 4I. Next, the aforementioned postdilatation may be effected as shown in FIG. 4J by positioning balloon 74 within stent 82 and expanding both. This procedure may further expand the stent, pushing it into adjacent plaque—helping to secure each.

Naturally, the balloon need not be reintroduced for postdilatation, but it may be preferred. Regardless, once the delivery device 80 and balloon catheter 72 are withdrawn as in FIG. 4K, the angioplasty and stenting procedure at the lesion in vessel 60 is complete. FIG. 4L shows a detailed view of the emplaced stent and the desired resultant product in the form of a supported, open vessel.

Furthermore, it is to be recognized that the subject invention may be practiced to perform “direct stenting.” That is, a stent may be delivered alone to maintain a body conduit, without preceding balloon angioplasty. Likewise, once one or more stents are delivered with the subject system (either by a single system, or by using multiple systems) the post-dilatation procedure(s) discussed above are merely optional. In addition, other endpoints may be desired such as implanting an anchoring stent in a hollow tubular body organ, closing off an aneurysm, delivering a plurality of stents, etc. In performing any of a variety of these or other procedures, suitable modification will be made in the subject methodology. The procedure shown is depicted merely because it illustrates a preferred mode of practicing the subject invention, despite its potential for broader applicability.

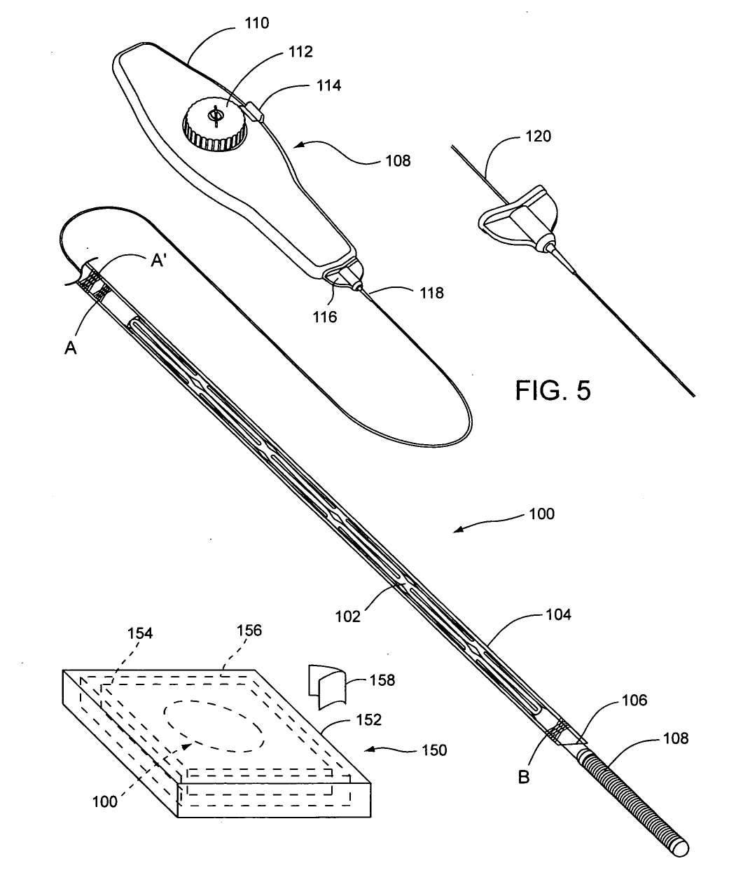

A more detailed overview of the subject delivery systems is provided in FIG. 5. Here, a delivery system 100 is shown along with a stent 102 held in a collapsed configuration upon the delivery guide member. A tubular member 104 is provided over and around the stent to restrain it from expanding. Tubular member 104 may include a canted or angled distal end 106 presenting a varying axial extent to effect a step-wise release of end segments of a stent. Such methodology is further described in U.S. patent application Ser. No. 10/967,079, entitled, “Delivery Guide Member Based Stent Anti-jumping Technologies.” At least some portion of tubular member 104 covering the stent comprises the hybrid or composite construction disclosed herein. Preferably, the entirety of the section of tubular member covering the stent constitutes the composite construction. For connecting this tube to a pull wire, the length of the composite tube will extent further. When the distal end of the tube comprises a portion of a simple sheath, then the composite section may be full-length or it may be limited to the section of the sheath subject to high radial loads by the stent. Still further, a hybrid approach may be employed where one of the patterns described below covers the stent portion of a simple sheath, and another pattern occupies a more proximal region (including the entire proximal region).

The tubular member employed to restrain the stent until removed therefrom to effect release may take of the form of a full-length sheath. For this purpose, the system may resemble those described in U.S. Pat. Nos. 6,280,465 or 6,833,003 or others. Alternatively, the composite tube used in the present invention may serve as a distal tubular restraint. In such instances, exemplary overall device construction approaches are provided in U.S. Pat. No. 6,736,839 or application Ser. Nos. 10/792,657, 10/792,679 and 10/792,684, filed on Mar. 2, 2004, and Ser. No. 10/991,721 filed Nov. 18, 2004. For their use in either approach, the referenced patents and applications are incorporated by reference herein in their entirety.

It is noted that the metalized portion of the sheath or tubular restraint need not run the entire length of the sheath. The metalized section of the tubular member need only cover a portion corresponding to the portion in apposition with at least a portion of the stent. For cost savings, complexity reduction, etc. such an approach may be especially advantageous in the case where the tubular member is used in a simple/full-length sheath.

The metal-reinforced section of the restraint will typically be offered to provide hoop strength for the tubular member to resist radial forces of the stent. In other instances, the metalized section will be provided not only to offer increased radial strength for a tubular member of a given thickness, but also to offer axial strength in the subject structure. In cases where the metalized portion is provided for axial strength, one or more metalized segments will typically form a proximal end of the tube wherein actuation force is applied either to the distal end of the tube or to some point therebetween.

In any case, the delivery guide preferably comprises a flexible atraumatic distal tip 108 of one variety or another. On the other end of the delivery device, a custom handle 110 may be provided. The device may include a lock 116. It may include various safety or stop features and or ratchet or clutch mechanisms to ensure one-way actuation. Furthermore, a removable interface member 118 may be provided to facilitate taking the handle off of the delivery system proximal end 120. The interface will be lockable with respect to the body and preferably includes internal features for disengaging the handle from the delivery guide. Once accomplished, it will be possible to attach or “dock” a secondary length of wire 122 on the delivery system proximal end, allowing the combination to serve as an “exchange length” guidewire, thereby facilitating changing-out the balloon catheter or performing another procedure. Alternatively, the wire may be an exchange-length wire.

FIG. 5 also shows packaging 150 containing at least one coiled-up delivery system 100. When a plurality of such systems are provided (in one package or by way of a number of packages held in stock), they are typically configured in support of a methodology where an appropriate one is picked to reach a target site and deploy a stent without unintended axial movement of the same as per the methodology of Ser. No. 10/792,684, referenced above. Thus, the packaging may serve the purpose of providing a kit or panel of differently configured delivery devices. In the alternative, the packaging may be configured as a tray kit for a single one of the delivery systems.

Either way, packaging may include one or more of an outer box 152 and one or more inner trays 154, 156 with peel-away coverings as is customary in packaging of disposable products provided for operating room use. Naturally, instructions for use 158 can be provided therein. Such instructions may be printed product or be provided in connection with another readable (including computer-readable) medium. The instructions may include provision for basic operation of the subject devices and associated methodology.

In support of such use, it is to be understood that various radiopaque markers or features may be employed in the system to 1) locate stent position and length, 2) indicate device actuation and stent delivery and/or 3) locate the distal end of the delivery guide. As such, platinum (or other radiopaque material) bands or other markers (such as tantalum plugs) may be variously incorporated into the system. Especially where the stent employed may shorten somewhat upon deployment, it may also be desired to align radiopaque features with the expected location (relative to the body of the guide member) of the stent upon deployment. For such purposes, radiopaque features may be set upon the core member of the delivery device proximal and distal of the stent at points A, A′ and B indicated, respectively.

Additionally, it is to be observed that the metalized restraint itself in many variations of the invention may be highly radiopaque. The degree of radiopacity will vary depending upon the metal selected for coating the underlying polymer. Furthermore, even if the tubular member is not adequately radiopaque by itself to offer clear visibility, then the cumulative effect of its radiopacity with that of the stent may offers desirable visibility.

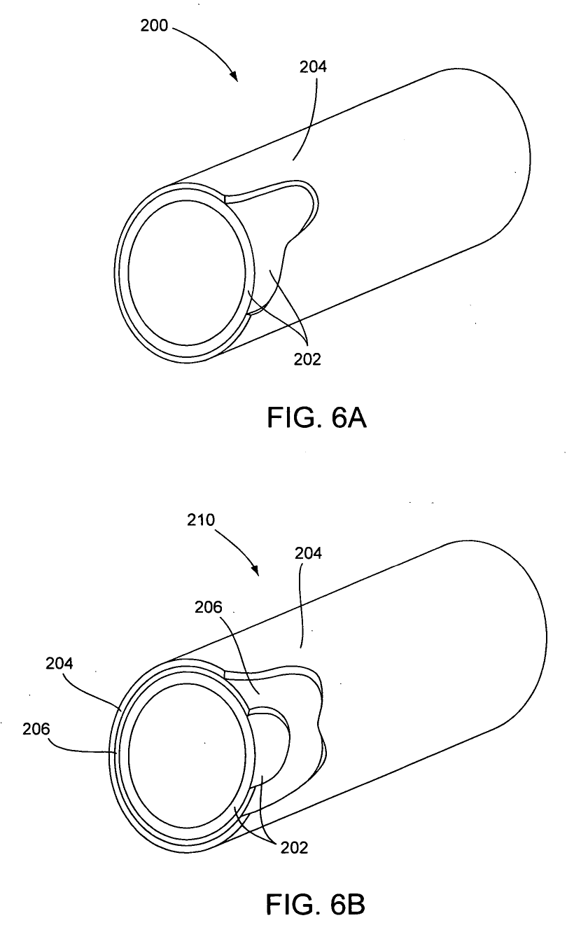

Turning now to FIG. 6A, it shows a cutaway perspective view a tubular member 200 according to the present invention. It includes an inner polymer layer 202 and an outer metal layer 204 set over the inner polymeric layer. The structure may be formed in the manner discussed above, or otherwise.

In FIG. 6B, a cutaway perspective view of another tubular 210 member is shown. In addition to an inner polymer layer 202 and outer metal layer 204, it includes and intermediate metal layer 206. As commented upon above, such a layer will typically be provided as a highly conductive substrate upon which to electroplate the outer layer.

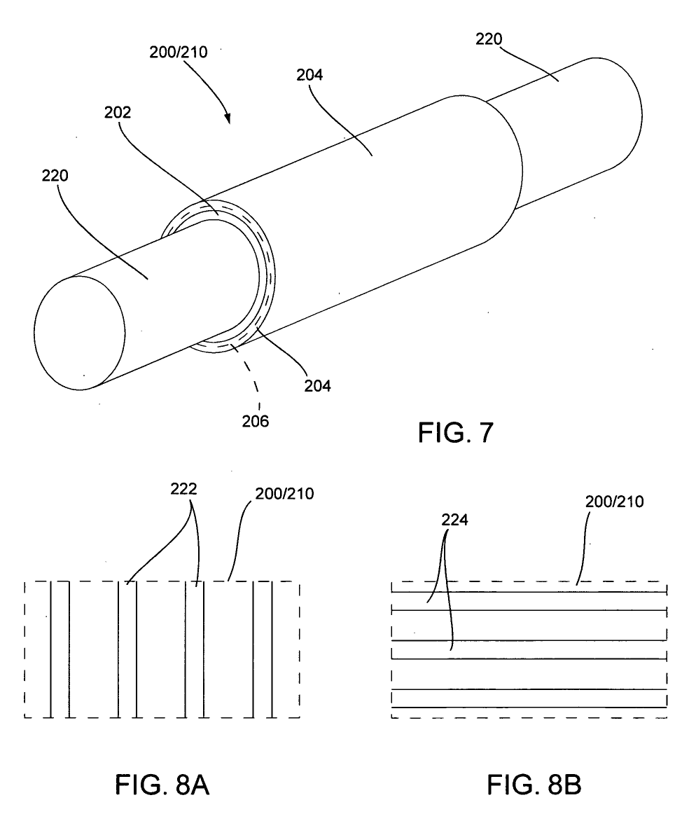

FIG. 7 shows a perspective view of tubular member 200/210 (as provided according to either of FIGS. 6A or 6B) at an intermediate stage of production. In the figure, inner polymer layer 202 is provided upon a mandrel 220. As discussed above, this mandrel will be removed—either physically withdrawn or etched away—to release the desired tubular structure.

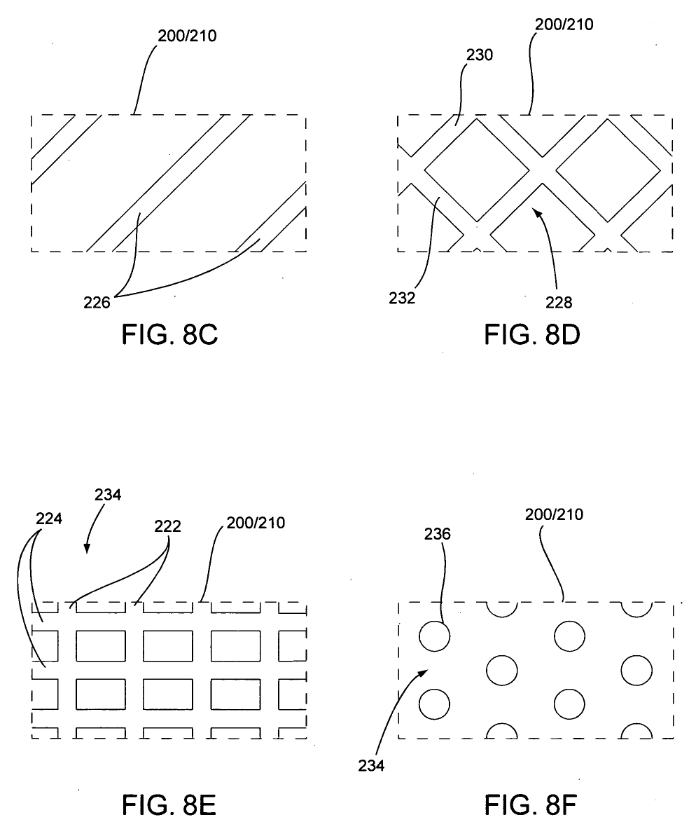

Other processing steps may be employed in the construction of the subject tubular member. These may derive from a desire to provide a more complex tubular metal structure than one that is simply coated with one or more layers of metal. Those with skill in the art will readily appreciate what is required to produce any of the structures shown in FIGS. 8A-8F. The structures where portion(s) of the tubular member are not covered by metal may be desired in order to, for example, increase flexibility to improve the trackability of a system including the subject tubing. In another approach, instead of selective metal coating, the entire structure may be coated and then openings formed therein.

Selective metal coating may be accomplished by masking techniques or other means. The tubing could have holes cut into it by laser cutting, EDM or other means.

In one manner or another, various coverage patterns can be achieved. FIGS. 8A-8F illustrate optional patterns upon “unrolled” tubular members. FIG. 8A shows a laid-open tube 200/210 with a series of metal bands 222. These may be oriented perpendicular to an axis of the tube as shown or at an angle. FIG. 8B shows a plurality of stripes 224 longitudinally oriented along the tube. FIG. 8C shows a spiral pattern 226. The “winding” angle shown is set at 45 degrees. However, other angles may be desired from a perspective of offering greater flexibility or longitudinal strength. FIG. 8D shows a grid or lattice 228 with segments 230, 232 set at ±45 degrees, respectively, relative to a longitudinal axis of the part. Naturally, the angles of the grid can, again, be modified to reach the desired performance characteristics. For example, FIG. 8E shows a 0-90 degree grid pattern 234 in which vertical and horizontal segments 222, 224 are employed. FIG. 8F shows a dimpled pattern 234 with openings 236 in the metal or the metal and the inner polymeric layer.

Naturally, in any of these variations. the parameters (i.e., thickness, width, diameter, etc.) of the sections defining the metalized portion(s) and/or complimentary parameters of the open sections (as formed by actual openings, bare substrate, or less thickly coated substrate) can be varied as desired. Depending on the task at hand, one configuration may be preferred to another. For instance, to maximize strength for a given diameter and wall thickness, a fully covered substrate will generally be preferred as shown in any of FIGS. 6A, 6B and 7. When a tubular member 200/210 is to be employed in a delivery system as a stent restraint and pulled upon to effect restraint withdrawal for stent delivery actuation, axial and circumferential strength of the tubular member is important. In such circumstances, the tubular member should also be configured to avoid substantially necking-down or reducing in diameter when placed in tension. For such purposes, a tubular member that is fully covered or employs one of the coverage patterns shown in FIGS. 8E or 8F may be desired.

In addition, it is contemplated that any of these coverage approaches (or others) may be employed in combination with each other along the length of the tubular member. The purpose of doing so may be to tune or adjust strength and bending or torsional stiffness along the length of the body.

Though not necessarily required (because the stent delivery system could be used in connection with a supplemental restraint or sheath for device storage), due to the metal coating of the tubular member, it will be able to resist creep. In contrast, many polymers —including PTFE—are prone to stretching under conditions of prolonged exposure to a force. Those configurations of the device best able to avoid diameter enlargement due to radial forces of a compressed stent include a tubular member that is fully covered and those employing one of the coverage patterns shown in FIGS. 8A, 8D, 8E and 8F that offer a radial component.

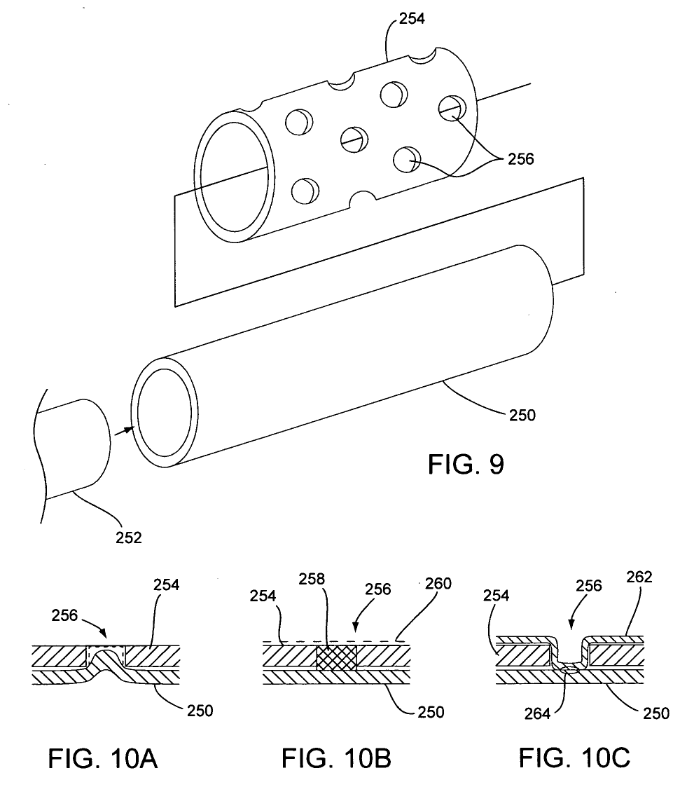

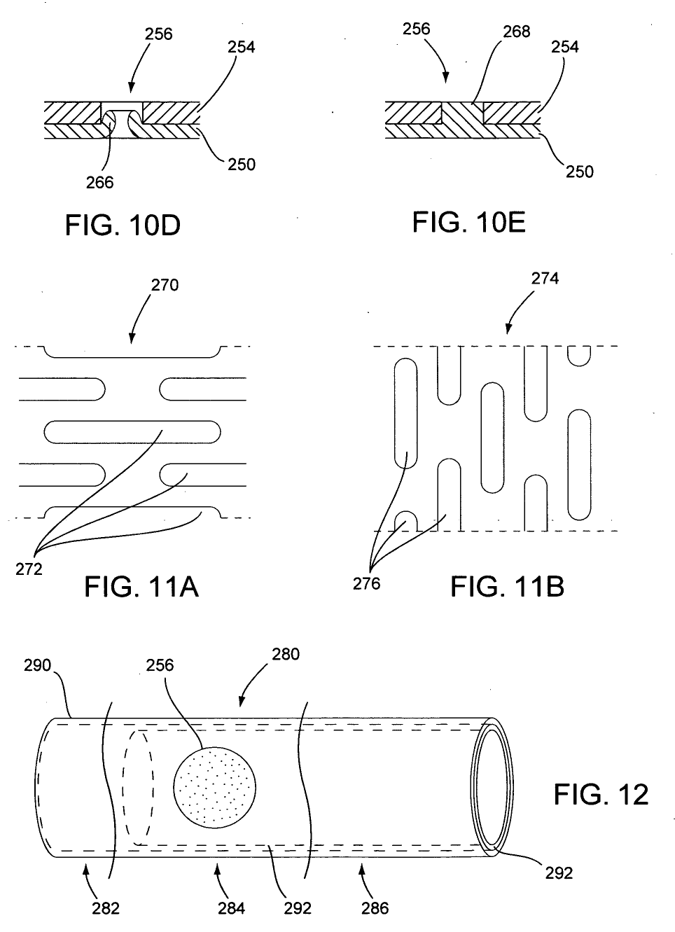

As for variation of the invention in which the composite tube is assembled from discrete components—as opposed to grown or built up—FIG. 9 show an example of an interlocking approach to composite tubular construction. A polymeric tubular member 250 is provided. It may be supported upon a mandrel 252 for insertion into metal/metallic tube 254 or it may be formed thereon as discussed above. When set within tube 254 as indicated, tube 250 and 254 are physically locked together. As referenced above, this may be a mechanical interlock between the members themselves with sections of tube 250 received within openings 256 formed in the wall of tube 254. Such an approach is shown in FIG. 10. Here, interior tube 250 is deformed outward to fill opening 256 of tube 254.

This result may be accomplished by plastically deforming tube 250 by pneumatic or fluid pressure applied therein. The fill factor may be partial as shown, or complete as indicated by dashed line. The latter condition would typically be achieved by performing the pressurization procedure with a sleeve (not shown) set about outer tube 256 in order to limit expansion of the polymeric material.

Such an approach may be desired from the perspective providing a construction that is smooth along its exterior in order to minimize traumatic potential or undesired frictional interaction with external components. FIG. 10 shows another approach in which a smooth or flush exterior surface is easily achieved. In this approach, the inner and outer tubes are interlocked by an intermediate structure 258. This “structure” may comprise glue or polymer welded to tube 250. Still further, plug 258 may be the remainder of a portion of material applied to the entire exterior surface of tube 254 by spraying, dip coating, heat shrinking, etc, that is later removed. Alternatively, that layer 260 may be left intact as indicated by the dashed lines.

FIG. 10C, shows another outside-in connection approach. In a similar fashion to the approach in FIG. 10, a layer of material is deformed to fill-in hole 256. Except, in the application shown in FIG. 10C, an outer layer 262 fills the hole as makes contacted with inner layer. They may be fused directly together, or an intermediate adhesive may be employed along material boundary 264 may be employed. Once the members are interconnected, layer 262 may be skived/trimmed off or left intact as shown.

FIG. 10D shows yet another approach to interconnecting the members forming the composite tube. Here, section of polymer tube 250 are melted to form a bead(s) 266 that fills hole(s) 256. Simple application of hot air by a heat gun or another method such as running a low current, high voltage arc between a conductive mandrel and one or more external electrodes (neither shown) may be employed be employed in this regard.

In FIG. 10E, the inner tube 250 is formed by collapsing the outer tube about it under heat and/or pressure to cause a plug 268 of material to form by a reduction in wall thickness of the inner material and flow into hole 256. Naturally, such a forming procedure would be accomplished with tube 250 set over a mandrel. As above, the mandrel may simply be removable, or comprise a sacrificial material.

FIGS. 11A and 11B show alternate alternative cut-out patterns for the metallic tubular member. Pattern 270 shown in FIG. 11A is optimized for expanding the tubular member and then collapsing it about an inner polymeric tube, for example, to achieve the sort of interlocking relationship of the metal and polymer tubes shown in FIG. 10E. In this example slits 272 having a primary longitudinal component can be employed to expand the tube in stent-like fashion. Pattern 274 shown in FIG. 11B offers slits 276 having a primary horizontal component. They are provided to offer increased flexibility while still offering good torque transmission characteristics. Note that either pattern may have a helix or stagger of units imparted to it in order to homogenize performance of its length. This feature is specifically illustrated in connection with pattern 274 shown in FIG. 11B.

FIG. 12 shows an example of yet another interlocking composite tubular construction. The figure illustrates a tube 280 including multiple different feature zones 282, 284, 286 (from proximal to distal end). Zone 282 is defined by metal tube 290 alone. (Though, this zone could, conversely, be defined by polymer tube 292 alone if the metal tube 290 terminates in zone 284 rather than the polymer tube.) In the former case, disinclusion of polymer tubing along the major length of the hypotube 290 simplifies construction. Specifically, it may be difficult to feed a long section of polymer tube within the metal tube.

Another unique feature of the multi-zone construction concerns intermediate zone 284. It is in this section—alone—that the tubes are interconnected. By moving interconnection or interlock features away from the stent-restraining region, an improved stent/restraint or sheath interface may be possible. Furthermore, the connection may be simplified in that a larger window 256 and fill section 258/264/266/268/etc. can be accommodated without interfering with region 286. In addition, the larger feature(s) may be more easily formed/constructed and/or more robust.

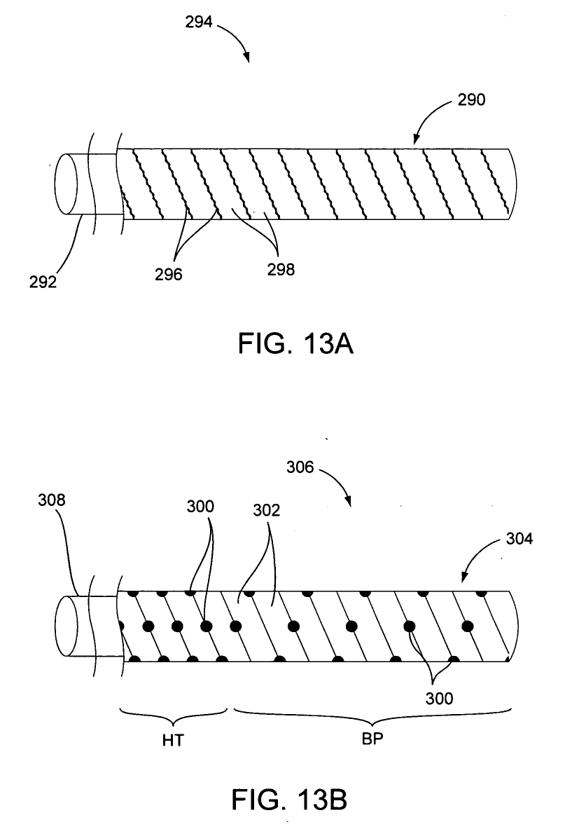

Finally, another approach tubular member construction is shown in FIGS. 13A and 13B. Here, metal ribbon 290 is wound about a polymer tube 292 set upon a mandrel (not shown) to form a composite tubular body 294. The seam between adjacent turns of the ribbon is welded, for example, by laser welding. Either a fully-wound structure is welded, or turns of the coil are welded as they are formed on the mandrel.

The metal ribbon wrapped around the mandrel and/or mandrel and polymer tube may have its edges set in abutment or overlap slightly. A slight overlap of material may be desirable from a welding and metal-flow perspective. Still, wrapping the coil turns so they simply abut one another may be desirable from the standpoint of minimizing tube diameter or external/internal variability once welded.

In any case, because very thin ribbon is used (e.g., on the order of 0.0002 to 0.0006 inch thick—with 5 to 20, generally 10 times the width) with very low heat capacity, the energy from the welding process can be localized such that it internal polymer is not burnt or otherwise damaged. The ribbon may comprise stainless steel, titanium, titanium alloy (including NiTi) or another material such at PtIr or PtW (e.g., for their relatively high strength and excellent radiopacity). Naturally, any of the other alloys noted above may alternatively be used as well.

In cases where overlapping ribbon is employed, the stacked layers of ribbon may offer further protection to underlying polymer during welding. Alternatively, interior spaces formed within the structure may offer interlock/interlocking sites for an expanded polymer tube inserted after the welding process.

Still, even if sections of the internal polymer material melt or flow during welding, because any such polymer will be set upon a mandrel no deleterious effect on internal surface finish may be observed. Indeed, even in sections (or a continuous spiral of material) is ablated, the relative amount of material lost may be of no substantial consequence to the overall lubricity provided by the interior polymer layer.

In any of these variations, the ribbon may be welded together in a continuous fashion or spot-welded. By “continuous” what is meant is that a contiguous weld bead is formed connecting adjacent turns of the coil. Such an approach is illustrated in FIG. 13A, in which weld bead 296 connects coil 290 turns 298 into a unitary body 294. Where “spot welding” is employed, any configuration of weld attachment points may be employed. In FIG. 13B, weld sections 300 attach the turns 302 of coil 304 to form tubular body 304 upon polymer layer 306. As shown, by varying the concentration and/or patterns of welds, different performance regions can be engineered. A high-tensile strength “HT” region is formed in one section where a bending performance optimized “BP” section is formed over another portion of the structure. Typically, the former (HT) section is oriented proximally to the latter (BP) section since not only is improved flexibility often a system requirement distally, but also, improved tensile strength is useful proximally in regions subject higher forces (unaffected, or less affected by frictional losses).

Two different zones are show, but others may be employed. Likewise, while the zones are shown constructed employing spot-welded zones, more continuous weld beads or zones may be employed. In other words, the structure may more closely resemble that shown in FIG. 13A, minus open sections along the junction between adjacent coil turns.

Still further, variation in the wind angle or pitch is contemplated. Likewise, variable width ribbon may be employed in winding the structure in order to offer different performance characteristics along the length of the subject tubular member.

Methods

The methods herein may be performed using the subject devices or by other means. The methods may all comprise the act of providing a suitable device. Such provision may be performed by the end user. In other words, the ‘providing” (e.g., a delivery system) merely requires the end user obtain, access, approach, position, set-up, activate, power-up or otherwise act to provide the requisite device in the subject method. Methods recited herein may be carried out in any order of the recited events which is logically possible, as well as in the recited order of events.

Variations

Exemplary aspects of the invention, together with details regarding material selection and manufacture have been set forth above. As for other details of the present invention, these may be appreciated in connection with the above-referenced patents and publication as well as generally known or appreciated by those with skill in the art.

The same may hold true with respect to method-based aspects of the invention in terms of additional acts as commonly or logically employed. In addition, though the invention has been described in reference to several examples, optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in the appended claims, the singular forms “a,” “an,” “said,” and “the” include plural referents unless the specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth n the claims. Stated otherwise, except as specifically defined herein, all technical and scientific terms used herein are to be given as broad a commonly understood meaning as possible while maintaining claim validity.

The breadth of the present invention is not to be limited by the examples provided and/or the subject specification, but rather only by the plain meaning of the claim terms employed. That being said, we claim:

1. A tube comprising:
an inner polymeric layer, and
an outer metallic layer,
wherein the tube has an outer diameter less than about 0.018 inches and a wall thickness of between about 0.0004 and about 0.002 inches, and
wherein the metallic layer has a thickness between about 0.0002 and about 0.0015 inches.

2. The tube of claim 1, further comprising a medial metallic layer between the outer metallic layer and the inner polymeric layer.

3. The tube of claim 2, wherein the medial metallic layer comprises a metal selected from aluminum, copper, silver, gold, platinum and palladium.

4. The tube of claim 3, wherein the outer metallic layer comprises a metal selected from Nickel, Nickel Sulfamate, High Phosphate Nickel, NiTi, NiCo and CoCr electroplated upon the inner metallic layer.

5. The tube of claim 1, wherein the outer metallic layer comprises a metal selected from stainless steel, Nickel and NiTi vacuum deposited upon the inner polymeric layer.

6. The tube of claim 1, wherein the outer diameter of the tube is about 0.017 inches or less.

7. The tube of claim 6, wherein the outer diameter is about 0.016 inches or less.

8. The tube of claim 7, wherein the outer diameter is about 0.015 inches or less.

9. The tube of claim 8, wherein the outer diameter is about 0.014 inches or less.

10. The tube of claim 1, wherein the polymeric layer has a thickness between about 0.0002 and about 0.001 inches.

11. The tube of claim 1, wherein the wall thickness is about 0.0015 inches or less.

12. The tube of claim 1, wherein the outer metallic. layer includes a plurality of coatings.

13. The tube of claim 12, wherein the coatings comprise different materials.

14. The tube of claim 13, without an inner metallic layer.

15. The tube of claim 1, consisting of the inner polymeric layer, and the outer metallic layer.

16. A tube made by a process comprising:
providing a polymeric tube having outer diameter less than about 0.018 inches and a wall thickness of between about 0.00025 and about 0.0015 inches; and
depositing a structural metallic layer having a thickness between about 0.0002 and about 0.001 inches over the polymeric tube.

17. The tube of claim 16, wherein the polymeric tube is provided by drawing tubing to size.

18. The tube of claim 16, wherein the polymeric tube is provided by depositing polymer upon a mandrel, and the process further comprises removing the mandrel after the depositing.

19. The tube of claim 18, wherein the mandrel is removed by etching it from within the polymeric tube.

20. The tube of claim 16, wherein the structural metallic layer is deposited directly upon the polymeric tube.

21. The tube of claim 16, wherein the process further comprises depositing a highly conductive metallic layer directly upon the polymeric tube and the structural metallic layer is deposited upon the conductive layer by electroplating.

22. A tube made by a process comprising:
providing a metallic tube having outer diameter less than about 0.018 inches;
providing a polymeric tube within the metallic tube;
forming openings in a wall of the tube;
interlocking the tubes.

23. The tube of claim 22, wherein the interlocking is accomplished by expanding at least some portion of the polymeric tube.

24. The tube of claim 22, wherein the interlocking is accomplished by collapsing the at least some portion of the metallic tube.

25. The tube of claim 22, wherein the interlocking is by bonding to the polymeric tube through at least some of the openings.

26. A tube made by a process comprising:
winding a ribbon around a mandrel to an outer diameter of less than about 0.018 inches;
welding the ribbon to form a metallic tube.

27. The tube of claim 26, wherein a polymer tube is provided within the metallic tube.

28. The tube of claim 27, wherein the polymer tube is provided over the mandrel prior to the winding.

29. The tube of claim 27, wherein the polymer tube set within the metallic tube after the welding.

30. A stent delivery system comprising:
an elongate shaft adapted to maintain an axial position of the stent, and a tube according claim 1, 22 or 26 to hold a stent in a radially compressed configuration until withdrawn from the stent.

31. The stent delivery system of claim 30, further comprising a stent held within the tube.

32. The stent delivery system of claim 31, wherein the stent is a self-expanding stent.

33. The stent delivery system of claim 32, wherein the stent comprises superelastic NiTi alloy.
